(19) United States
(12) Patent Application Publication
(10) Pub. No.: US 2020/0251808 A1
(43) Pub. Date: Aug. 6, 2020

(54) RECEIVING UNIT AND PROCESSING SYSTEM

(71) Applicant: OLYMPUS CORPORATION, Tokyo (JP)

(72) Inventors: Shohei NISHI, Tokyo (JP); Takeshi NISHIYAMA, Tokyo (JP)

(73) Assignee: OLYMPUS CORPORATION, Tokyo (JP)

(21) Appl. No.: 16/855,280

(22) Filed: Apr. 22, 2020

Related U.S. Application Data
(63) Continuation of application No. PCT/JP2018/036750, filed on Oct. 1, 2018.

(30) Foreign Application Priority Data
Nov. 17, 2017 (JP) 2017-221651

(51) Int. Cl. H01Q 1/27; H04N 5/235; H04N 5/225; H04N 5/232; A61B 1/00; A61B 1/04

(52) U.S. Cl. CPC H01Q 1/273; H04N 5/2352; H04N 5/2256; H04N 2005/2255; A61B 1/00016; A61B 1/041; A61B 1/00057; H04N 5/23216

(57) ABSTRACT

A receiving unit includes: a receiving antenna configured to receive a wireless signal transmitted from a capsule endoscope introduced into a subject, the receiving antenna being attached to a body surface of the subject; a detector configured to detect a pressure between the receiving antenna and the subject; and a processor including hardware, the processor being configured to determine an attachment state of the receiving antenna to the body surface based on the pressure, and output a determination result of the attachment state.

### CROSS-REFERENCE TO RELATED APPLICATION

This application is a continuation of PCT International Application No. PCT/JP2018/036750 filed on Oct. 1, 2018, which designates the United States, incorporated herein by reference, and which claims the benefit of priority from Japanese Patent Application No. 2017-221651, filed on Nov. 17, 2017, incorporated herein by reference.

### BACKGROUND

### 1. Technical Field

The present disclosure relates to a receiving unit including a receiving antenna that receives a wireless signal transmitted from a capsule endoscope introduced into a subject and to a processing system including the receiving unit.

### 2. Related Art

In the related art, endoscopes are widely used as a medical observation device that is introduced into the body of the subject, such as a patient, to observe the inside of the body of the subject. In recent years, capsule endoscopes have been developed as a swallowable radio-wave generator having a capsule-shaped casing and including, for example, an imaging device and a communication device that wirelessly transmits the image data captured by the imaging device to the outside of the body. Capsule endoscopes have the function to, after the capsule endoscope is swallowed through the mouth of the patient to observe the inside of the subject's body, move in accordance with a peristaltic action inside an organ, such as esophagus, stomach, or small intestine, so as to sequentially execute capturing until the capsule endoscope is naturally excreted from the subject.

During the movement inside the subject, the image data captured by the capsule endoscope is sequentially transmitted to the outside of the body via wireless communications and stored in a memory provided inside or outside a receiving device via an antenna unit outside the body or presented on a display provided in the receiving device. A user, such as a doctor or a nurse, loads the image data stored in the memory into an information processing device via a cradle into which the receiving device is inserted and executes observation or diagnosis based on the image presented on the display of the information processing device or the position of the capsule endoscope when the image data is captured.

An antenna unit includes a plurality of receiving antennas that receive the image data wirelessly transmitted from the capsule endoscope. Each of the receiving antennas is secured to the body surface of the subject. As a technology that may be applied to the detection of the contact between the receiving antenna and the subject to check the attachment of the receiving antenna to the body surface, there is a known technology for detecting the contact between the body surface and the receiving antenna based on a change in impedance (see, for example, Japanese Laid-open Patent Publication No. 2000-216610).

### SUMMARY

In some embodiments, a receiving unit includes: a receiving antenna configured to receive a wireless signal transmitted from a capsule endoscope introduced into the subject, the receiving antenna being attached to a body surface of the subject; a detector configured to detect a pressure between the receiving antenna and the subject; and a processor comprising hardware, the processor being configured to determine an attachment state of the receiving antenna to the body surface based on the pressure, and output a determination result of the attachment state.

In some embodiments, a processing system includes: a receiving unit configured to receive a wireless signal transmitted from a capsule endoscope introduced into a subject to acquire an image; and a processing device configured to process the image. The receiving unit includes: a receiving antenna configured to receive the wireless signal, the receiving antenna being attached to a body surface of the subject; a detector configured to detect a pressure between the receiving antenna and the subject; and a processor comprising hardware, the processor being configured to determine an attachment state of the receiving antenna to the body surface based on the pressure, and identify the wireless signal corresponding to an acquisition time of a pressure for which the processor determines a poor attachment state. The processing device includes: a first storage configured to store the pressure, the image, and a determination result in association with one another, the pressure, the image, and the determination result being acquired from the receiving unit; and a controller configured to cause a first display to display the determination result and the image corresponding to the determination result.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic view illustrating a schematic configuration of a capsule endoscope system according to a first embodiment of the disclosure;

FIG. 2 is a block diagram illustrating a schematic configuration of the capsule endoscope system according to the first embodiment of the disclosure;

FIG. 3 is a block diagram illustrating a schematic configuration of a receiving system included in the capsule endoscope system according to the first embodiment of the disclosure;

FIG. 4 is a diagram illustrating a schematic configuration of a receiving antenna included in the capsule endoscope system according to the first embodiment of the disclosure;

FIG. 5 is a flowchart illustrating an antenna attachment checking process performed by the capsule endoscope system according to the first embodiment of the disclosure;

FIG. 6 is a diagram illustrating an example of a notification mode by a receiving device included in the capsule endoscope system according to the first embodiment of the disclosure;

FIG. 7 is a diagram illustrating an example of the notification mode by the receiving device included in the capsule endoscope system according to a modification of the first embodiment of the disclosure;

FIG. 8 is a diagram illustrating an example of the notification mode by the receiving device included in the capsule endoscope system according to the modification of the first embodiment of the disclosure;

FIG. 9 is a schematic view illustrating a schematic configuration of a capsule endoscope system according to a second embodiment of the disclosure;

FIG. 10 is a diagram illustrating a schematic configuration of the receiving antenna included in the capsule endoscope system according to the second embodiment of the disclosure;

FIG. 11 is a diagram illustrating a schematic configuration of the receiving antennas included in a capsule endoscope system according to a modification of the second embodiment of the disclosure;

FIG. 12 is a flowchart illustrating an antenna attachment checking process performed by the capsule endoscope system according to a third embodiment of the disclosure;

FIG. 13 is a flowchart illustrating an antenna attachment checking process performed by the capsule endoscope system according to a fourth embodiment of the disclosure;

FIG. 14 is a diagram illustrating an example of the notification mode by the receiving device included in the capsule endoscope system according to a fifth embodiment of the disclosure;

FIG. 15 is a diagram illustrating an example of the notification mode by the receiving device included in the capsule endoscope system according to the fifth embodiment of the disclosure;

FIG. 16 is a diagram illustrating an example of a display screen of a display device included in the capsule endoscope system according to a sixth embodiment of the disclosure; and

FIG. 17 is a diagram illustrating an example of the display screen of the display device included in the capsule endoscope system according to the sixth embodiment of the disclosure.

### DETAILED DESCRIPTION

A capsule endoscope system using a capsule endoscope for medical use is described below according to embodiments of the disclosure. The same components are denoted by the same reference numeral in the description of the drawings. It should be noted that the drawings are schematic and the relation between members in thickness and width, the ratio between members, and the like, differ from reality.

### First Embodiment

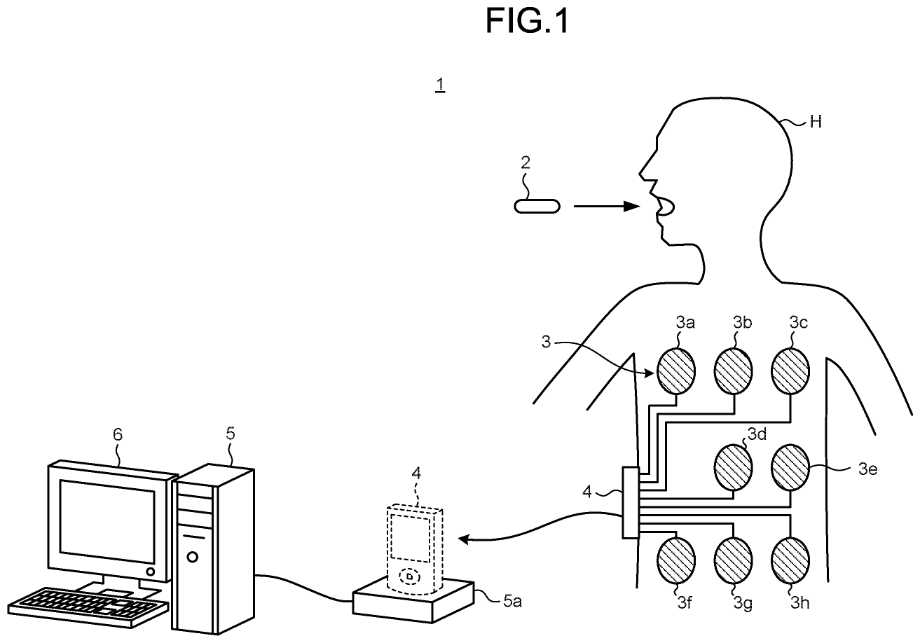

FIG. 1 is a schematic view illustrating a schematic configuration of a capsule endoscope system 1 according to a first embodiment of the disclosure. As illustrated in FIG. 1, the capsule endoscope system 1 according to the first embodiment includes: a capsule endoscope 2 that is a radio transmitting device that is inserted into a subject H, captures the inside of the subject H to generate image data, superimposes the image data on a wireless signal, and transmits it via a radio wave; a receiving device 4 that receives a wireless signal transmitted from the capsule endoscope 2 via a receiving antenna unit 3 including a plurality of receiving antennas 3a to 3h attached to the subject H; and a processing device 5 that fetches the image data generated by the capsule endoscope 2 from the receiving device 4 via a cradle 5a, processes the image data, and generates the image of the inside of the subject H. Images generated by the processing device 5 are output for display by, for example, a display device 6. In the description according to the first embodiment, at least one of the receiving antennas 3a to 3h and the receiving device 4 constitute a receiving unit.

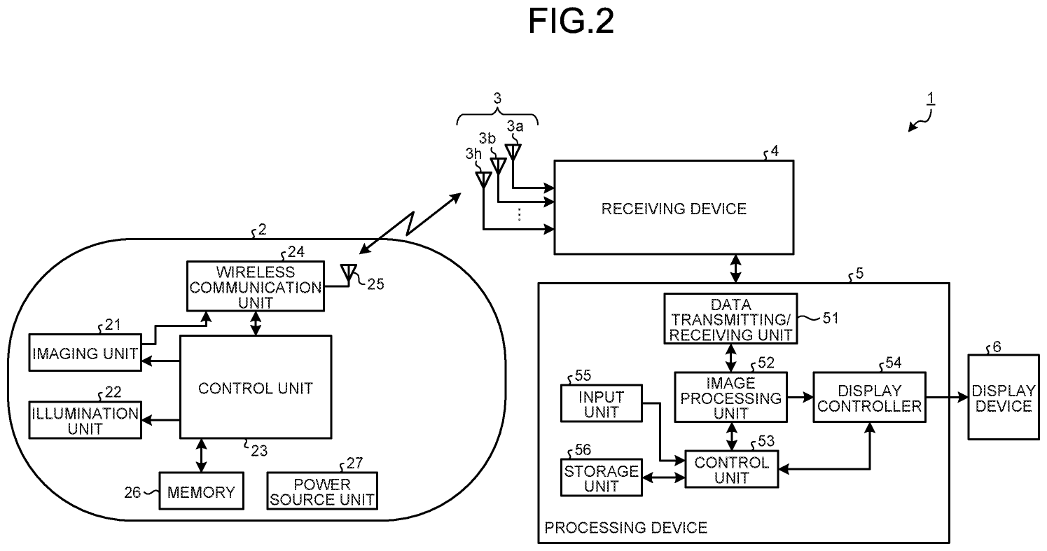

FIG. 2 is a block diagram illustrating a schematic configuration of the capsule endoscope system 1 according to the first embodiment of the disclosure. The capsule endoscope 2 includes an imaging unit 21, an illumination unit 22, a control unit 23, a wireless communication unit 24, an antenna 25, a memory 26, and a power source unit 27. The capsule endoscope 2 is an apparatus that has each of the above-described components built in a capsule-shaped casing having such a size that it may be swallowed by the subject H.

The imaging unit 21 includes, for example, an imaging element that generates, from an optical image formed on the light receiving surface, the image data that captures the inside of the subject H and outputs the image data; and an optical system, such as an objective lens, disposed on the light receiving surface side of the imaging element. The imaging element includes a CCD (charge coupled device) imaging element or a CMOS (complementary metal oxide semiconductor) imaging element, and includes a plurality of pixels arranged in a matrix to receive light from the subject H and conduct photoelectric conversion on the light received by the pixel to generate image data. The imaging unit 21 reads the pixel value from each horizontal line with regard to the pixels arranged in a matrix and generates the image data including multiple pieces of line data in which a synchronization signal is attached to each of the horizontal lines.

The illumination unit 22 includes a white LED, or the like, which generates white light that is illumination light. A configuration may be such that, instead of a white LED, white light is generated by combining lights from multiple LEDs, laser light sources, or the like, having different output wavelength bands, or a configuration may be such that a xenon lamp, a halogen lamp, or the like, is used.

The control unit 23 controls operation processing of each component of the capsule endoscope 2. For example, when the imaging unit 21 performs an imaging process, the control unit 23 controls the imaging unit 21 so as to perform the exposure and the reading processes on the imaging element and controls the illumination unit 22 so as to emit illumination light in accordance with the exposure timing of the imaging unit 21. The control unit 23 is configured by using a general-purpose processor, such as a CPU (central processing unit), or a dedicated processor such as various arithmetic circuits performing a specific function, e.g., ASIC (application specific integrated circuit).

The wireless communication unit 24 processes image data output from the imaging unit 21. The wireless communication unit 24 performs A/D conversion and predetermined processing on the image data output from the imaging unit 21, acquires digital-format image data, superimposes the image data on a wireless signal together with relevant information, and transmits it via the antenna 25 to an external unit. The relevant information includes, for example, identification information (e.g., serial number) assigned to identify the individual capsule endoscope 2. The wireless communication unit 24 is configured by using a dedicated processor such as various arithmetic circuits to perform a specific function, e.g., an ASIC. The antenna 25 is configured by using a pattern antenna, etc.

The memory 26 stores an execution program and a control program for the control unit 23 to perform various operations. The memory 26 may temporarily store image data, or the like, on which signal processing has been performed by the wireless communication unit 24. The memory 26 configured by using a RAM (random access memory), a ROM (read only memory), or the like.

The power source unit 27 includes: a battery that is a button battery, or the like; a power circuit that supplies the power to each unit; and a power switch that switches the on/off state of the power source unit 27, and the power source unit 27 supplies the electric power to each unit of the capsule endoscope 2 after the power switch is turned on. The power switch is a reed switch whose on/off state is switched due to, for example, an external magnetic force; before the capsule endoscope 2 is used (before the capsule endoscope 2 is swallowed by the subject H), the power switch is switched to the on state due to the application of a magnetic force to the capsule endoscope 2 from outside.

After the capsule endoscope 2 described above is swallowed by the subject H, the capsule endoscope 2 moves within a digestive tract of the subject H due to a peristaltic motion of an organ, or the like, to sequentially capture an in-vivo site (esophagus, stomach, small intestine, large intestine, and the like) in a predetermined cycle (e.g., the cycle of 0.5 seconds). Then, the image data acquired during the imaging operation together with relevant information are sequentially transmitted with radio waves to the receiving device 4 via the receiving antenna unit 3. In this case, the capsule endoscope 2 alternately transmits the wireless signal including the image data and the relevant information and the wireless signal for position detection.

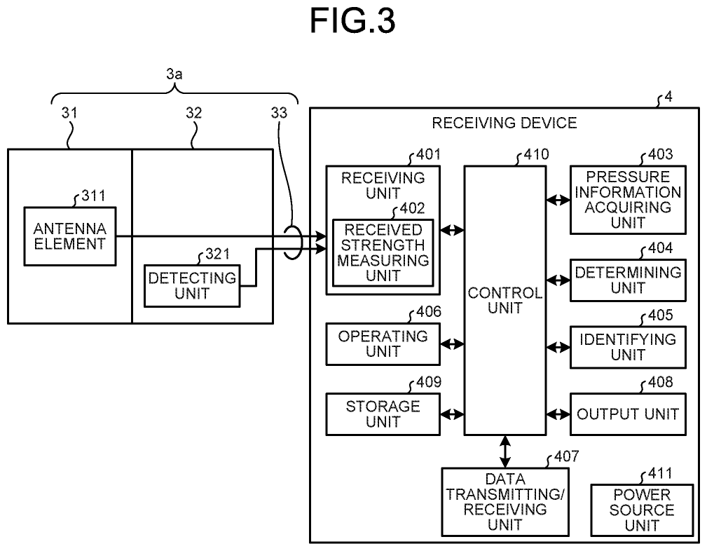

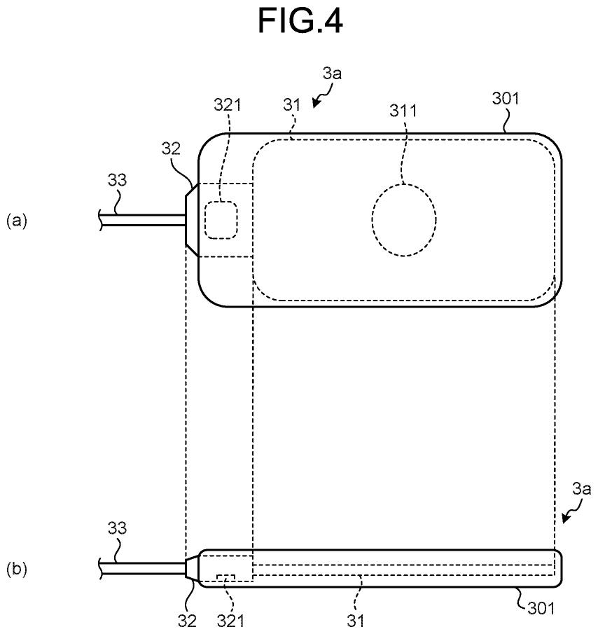

FIG. 3 is a block diagram illustrating a schematic configuration of a receiving system included in the capsule endoscope system 1 according to the first embodiment of the disclosure. FIG. 4 is a diagram illustrating a schematic configuration of a receiving antenna (the receiving antenna 3a) included in the capsule endoscope system 1 according to the first embodiment of the disclosure. In (a) and (b) of FIG. 4, the receiving antenna 3a is viewed in directions perpendicular to each other. With reference to FIG. 3, the connection mode between the receiving antenna 3a and the receiving device 4 is described as an example. The receiving antenna 3a includes: an element mounting section 31 on which an antenna element 311 is mounted, which is secured to a predetermined position of the subject H to receive a wireless signal from the capsule endoscope 2; a breakage preventing section 32 that is coupled to an end of the element mounting section 31 to prevent the breakage of the element mounting section 31; and a cable 33 for putting together the signal lines extending from the breakage preventing section 32 and the element mounting section 31 via the breakage preventing section 32.

The antenna element 311 receives a wireless signal transmitted from the capsule endoscope 2 and outputs the wireless signal to the receiving device 4. The antenna element 311 is configured by using a loop antenna or a dipole antenna.

The breakage preventing section 32 includes a detecting unit 321 that is provided on a surface at the body surface side (the side facing the body surface) when attached to the subject H to detect the pressure applied to the body surface by the receiving antenna 3a. The detecting unit 321 converts the detected pressure into an electrical signal to generate the pressure information and outputs the pressure information to the receiving device 4. The detecting unit 321 is configured by using a piezoelectric element or a load cell. In the configuration illustrated in FIG. 4, when the receiving antenna 3a is attached to the subject H, the detecting unit 321 is located closer to the body surface side than the receiving antenna 3a.

One end of the cable 33 is coupled to the breakage preventing section 32, and the other end thereof is coupled to the receiving device 4. In the first embodiment, the receiving antennas 3a to 3h are denoted by numbers 1 to 8, sequentially starting from the receiving antenna 3a.

The element mounting section 31 and the breakage preventing section 32 are covered with a cover (securing part) 301 (see FIG. 4). The cover 301 includes a known securing means, such as an adhesive sheet, on the outer surface at the side where the detecting unit 321 is exposed. The cover 301 is secured to a predetermined position on the body surface of the subject H with the securing means.

Although the configuration of the receiving antenna 3a has been described as an example, the receiving antennas 3b to 3h have the same configuration.

The receiving device 4 includes a receiving unit 401, a pressure information acquiring unit 403, a determining unit 404, an identifying unit 405, an operating unit 406, a data transmitting/receiving unit 407, an output unit 408, storage unit 409, a control unit 410, and a power source unit 411.

The receiving unit 401 receives image data and relevant information, which are wirelessly transmitted from the capsule endoscope 2, via the receiving antenna unit 3 including the multiple (eight in FIG. 1) receiving antennas 3a to 3h. The receiving unit 401 includes a received strength measuring unit 402 that measures the received strength (RSSI: Received Signal Strength indicator) of a wireless signal received by the receiving antennas 3a to 3h. The receiving unit 401 selects the antenna with the highest received strength from the receiving antennas 3a to 3h based on the received strengths measured by the received strength measuring unit 402 and transmits the wireless signal received by the selected antenna for image generation to the processing device The receiving unit 401 is configured by using a processor such as a CPU or an ASIC and executes predetermined signal processing, such as demodulation processing or A/D conversion, on the received image signal.

The received strength measuring unit 402 measures the received strength with regard to each of the receiving antennas 3a to 3h when the receiving unit 401 receives a wireless signal. The storage unit 409 may store all the measured received strengths and the image data received the receiving unit 401 in association with each other. The received strength measuring unit 402 is configured by using a processor such as a CPU or an ASIC.

The pressure information acquiring unit 403 acquires the pressure information generated by the detecting unit 321. Specifically, the pressure information acquiring unit 403 acquires the pressure information generated by each of the receiving antennas 3a to 3h. The pressure information acquiring unit 403 may store the acquired pressure information in the storage unit 409. The pressure information acquiring unit 403 is configured by using a processor such as a CPU or an ASIC.

The determining unit 404 uses the pressure information on the receiving antennas 3a to 3h, input from the pressure information acquiring unit 403, to determine (the attachment state) whether each of the receiving antennas 3a to 3h is properly secured to the subject H. Specifically, the determining unit 404 compares the pressure information on each of the receiving antennas 3a to 3h with the preset threshold to determine whether there is a receiving antenna that is not properly secured. The threshold used here is set based on the pressure that is detected when a receiving antenna is properly secured. The determining unit 404 outputs a determination result to the identifying unit 405 via the control unit 410. The determining unit 404 is configured by using a processor such as a CPU or an ASIC.

When it is determined that there is a receiving antenna that is not properly secured in accordance with a determination result of the determining unit 404, the identifying unit 405 identifies the receiving antenna. Specifically, the identifying unit 405 extracts the pressure information in which the detected pressure is less than the threshold and identifies the receiving antenna corresponding to the pressure information. The identifying unit 405 is configured by using a processor such as a CPU or an ASIC.

The operating unit 406 is an input device (input unit) used by the user to input various setting information and instruction information to the receiving device 4. The operating unit 406 receives an input instruction signal regarding settings, etc. The operating unit 406 is, for example, a switch, a button, or a touch panel provided on the control panel of the receiving device 4.

The data transmitting/receiving unit 407 transmits image data and relevant information stored in the storage unit 409 to the processing device 5 when the data transmitting/receiving unit 407 is communicatively connected to the processing apparatus 5. The data transmitting/receiving unit 407 is configured by using a communication I/F such as a USB or a LAN.

The output unit 408 displays images, outputs sound or light, and generates vibrations. The output unit 408 displays the image obtained after predetermined image processing has been performed on the image data stored in the storage unit 409, and generates sound, light, or vibration. The output unit 408 is configured by using at least one of a display such as a liquid crystal display or an organic EL display, a speaker, a light source such as an LED, and a vibration generator such as a vibration motor. According to the first embodiment, a display, a speaker, and an LED constitute the output unit 408.

The storage unit 409 stores programs for operating the receiving device 4 to perform various functions, a threshold for determination, image data and relevant information received by the receiving device 4, etc. The storage unit 409 is configured by using a RAM, a ROM, etc.

The control unit 410 reads various programs stored in the storage unit 409 to transfer instructions, data, and the like, to each unit included in the receiving device 4 and controls the overall operation of the receiving device 4 in an integrated manner. The control unit 410 configured by using a processor such as a CPU or an ASIC.

The power source unit 411 supplies electric power to each unit of the receiving device 4. The power source unit 411 is configured by using a battery such as an electric battery.

The receiving device 4 of this type is attached to and carried by the subject H while the capsule endoscope 2 conducts capturing, e.g., while the capsule endoscope 2 is swallowed by the subject H, is passed through a digestive tract, and is then discharged. In this period, the receiving device 4 stores image data and relevant information received via the receiving antenna unit 3 in the storage unit 409.

After the capturing by the capsule endoscope 2 is finished, the receiving device 4 is removed from the subject H and is set in the cradle 5a (see FIG. 1) coupled to the processing device 5. Thus, the receiving device 4 is communicatively connected to the processing device 5 so as to transmit (download) image data and relevant information stored in the storage unit 409 to the processing device 5.

Referring back to FIG. 2, the processing device 5 is configured by using, for example, a workstation including the display device 6 such as a liquid crystal display. The processing device 5 includes a data transmitting/receiving unit 51, an image processing unit 52, a control unit 53, a display controller 54, an input unit 55, and a storage unit 56.

The data transmitting/receiving unit 51 is coupled to the receiving device 4 via the cradle 5a to transmit/receives data to/from the receiving device 4. The data transmitting/receiving unit 51 is configured by using a communication I/F such as a USB and a LAN.

The image processing unit 52 reads a predetermined. program stored in the storage unit 56 described later to execute predetermined image processing on the image data input from the data transmitting/receiving unit 51 so as to generate information useful for observation or diagnosis. The image processing unit 52 is configured by using a general-purpose processor such as a CPU or a dedicated processor such as various arithmetic circuits, e.g., an ASIC, for executing a specific function.

The control unit 53 reads various programs stored in the storage unit 56 to for example transfer a command or data to each unit included in the processing device 5 based on a signal input via the input unit 55 or image data input from the data transmitting/receiving unit 51 so as to control the overall operation of the processing device 5 in an integrated manner. The control unit 53 is configured by using a general-purpose processor such as a CPU or a dedicated processor such as various arithmetic circuits, e.g., an ASIC, for executing a specific function.

The display controller 54 causes the display device 6 to display the image generated after predetermined processing such as gradation processing or data decimation in accordance with the display range of an image in the display device 6 is performed on image data. The display controller 54 is configured by using a general-purpose processor such as a CPU or a dedicated processor such as various arithmetic circuits, e.g., an ASIC, for executing a specific function.

The input unit 55 receives input of information or a command corresponding to the user's operation. The input unit 55 is configured by using an input device such as a keyboard, a mouse, a touch panel, or various switches.

The storage unit 6 stores programs for performing various functions by operating the processing device 5, various types of information used while the program is executed, image data and relevant information acquired via the receiving device 4, in-vivo images generated by the image processing unit 52, etc. The storage unit 56 is configured by using, for example, a semiconductor memory such as a flash memory, RAM, or ROM, a recording medium such as an HOD, MO, CD-R, or DVD-R, and a drive device that drives the recording medium.

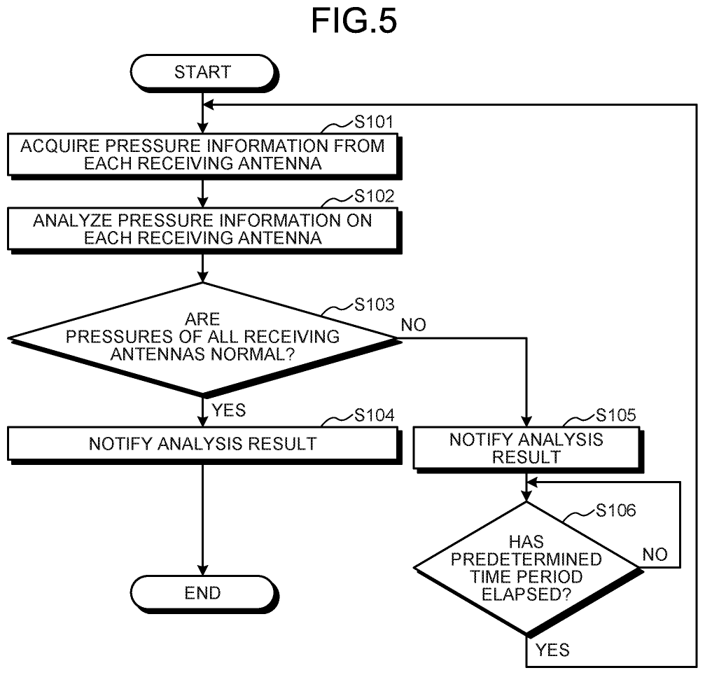

Next, with reference to FIG. 5, a process for checking whether the receiving antennas 3a to 3h are properly attached to the subject H is described. FIG. 5 is a flowchart illustrating an antenna attachment checking process performed by the capsule endoscope system 1 according to the first embodiment of the disclosure. In the description below, each unit operates under the control of the control unit 410.

After the receiving device 4 is activated, the detecting unit 321 generates the pressure information, and the pressure information acquiring unit 403 acquires the pressure information generated by the detecting unit 321 (Step S101). The pressure information acquiring unit 403 acquires the generated pressure information with regard to each of the receiving antennas 3a to 3h.

Subsequently, the determining unit 404 and the identifying unit 405 analyze the pressure information (Step S102). First, the determining unit 404 compares the pressure information on each of the receiving antennas 3a to 3h with the threshold to determine whether there is a receiving antenna whose pressure is less than the threshold. Then, when the determining unit 404 determines that there is a receiving antenna whose pressure is less than the threshold, the identifying unit 405 identifies the corresponding receiving antenna based on the pressure information. The identifying unit 405 outputs, to the control unit 410, the analysis result in which the identified receiving antenna is related to the information indicating that there is a receiving antenna whose pressure is not appropriate. Conversely, when it is determined that there is no receiving antenna whose pressure is less than the threshold, the identifying unit 405 outputs, to the control unit 410, the analysis result that is the information indicating that the pressures of all the receiving antennas are normal.

At Step S103 after Step S102, the control unit 410 determines whether the pressures of all the receiving antennas 3a to 3h are normal based on the analysis results. When it is determined that the pressures of all the receiving antennas 3a to 3h are normal according to the analysis result (Step S103: Yes), the control unit 410 proceeds to Step S104.

At Step S104, the control unit 410 causes the output unit 408 to notify the analysis result. The output unit 408 generates sound and presents, on the display, the information indicating that the receiving antennas 3a to 3h are properly attached or causes the LED to emit light in the color indicating the normal.

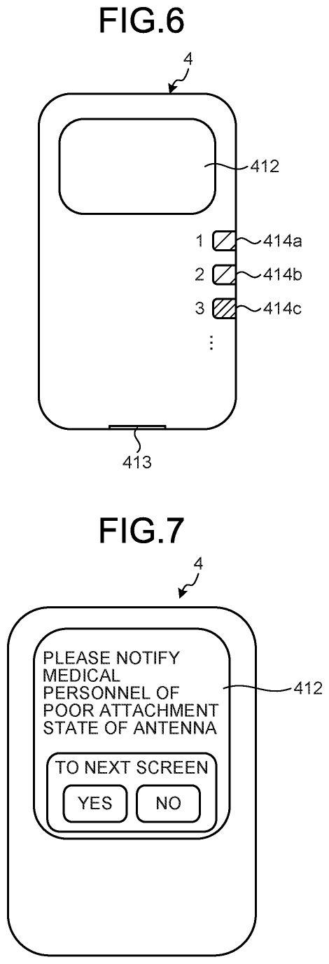

FIG. 6 is a diagram illustrating an example of the notification mode by the receiving device 4 included in the capsule endoscope system 1 according to the first embodiment of the disclosure. As illustrated in FIG. 6, the receiving device 4 includes a display section 412, a sound output section 413, and a plurality (eight according to the first embodiment) light emitting sections 414a, 414b, 414c, . . . . In the case of Step S104, the display section 412 displays the information indicating that the pressures of all the receiving antennas 3a to 3h are normal. The sound output section 413 generates sound, e.g., beep sound, to notify that the analysis result is being output. The number of the light emitting sections 414a, 414b, 414c, . . . , correspond to the number of the receiving antennas 3a to 3h (in FIG. 6, the number of the corresponding receiving antenna is described on the side of each of the light emitting sections). In the case of Step S104, all the light emitting sections emit light in the same color (e.g., green) indicating the normal. In a case where a vibration generator is provided instead of the sound output section 413, the control unit 410 causes the output unit 408 to generate vibrations at the time of notification.

After Step S104 is executed, the control unit 410 terminates the process of checking the attachment of the receiving antennas 3a to 3h. Specifically, the control unit 410 terminates the process of checking the attachment when the instruction signal indicating the termination of the attachment checking has been input via the operating unit 406 or when there is no input of the instruction signal during the set time.

Conversely, at Step S103, when it is determined that there is a receiving antenna whose pressure is not normal in accordance with an analysis result (Step S103: No) , the process proceeds to Step S105.

At Step S105, the control unit 410 causes the output unit 408 to notify the analysis result. In this case, the display section 412 displays the information indicating that there is a receiving antenna whose pressure is not normal. The sound output section 413 generates sound, e.g., beep sound, indicating that the analysis result is being displayed. A configuration may be such that, among the light emitting sections 414a, 414b, 414c, . . . , the light emitting section for the receiving antenna whose pressure is not normal emits light in a color (e.g., yellow) different from the color indicating the normal.

At Step S106 after Step S105, the control unit 410 determines whether a predetermined time period has elapsed after the previous pressure information is acquired. The predetermined time period is set based on, for example, the time it takes to reattach the receiving antenna. When it is determined that the predetermined time period has not elapsed (Step S106: No), the control unit 410 repeatedly checks the elapsed time. Conversely, when it is determined that the predetermined time period has elapsed (Step S106: Yes), the control unit 410 returns to Step S101 to repeat the above-described process.

As described above, after the receiving antenna is attached to the subject H and the receiving device 4 is activated, the attachment checking process is automatically executed, and the notification process is continued until the receiving antenna is properly attached.

According to the above-described first embodiment, the detecting unit 321 is provided to detect the pressure against the subject with regard to each of the receiving antennas, and the receiving device 4 conducts analysis as to whether the receiving antenna is properly attached or which receiving antenna is not properly attached in accordance with the result of detection by the detecting unit 321. Thus, it is possible to detect whether a receiving antenna is properly secured to the subject H. According to the first embodiment, the subject H or the user is prompted to properly attach the receiving antenna before examination and therefore the examination by the capsule endoscope 2 may be executed while the receiving antenna is properly attached.

In the description according to the above-described first embodiment, a notification is given even when the receiving antenna is properly attached; however, the notification process may be omitted when the receiving antenna is properly attached.

### Modification of the First Embodiment

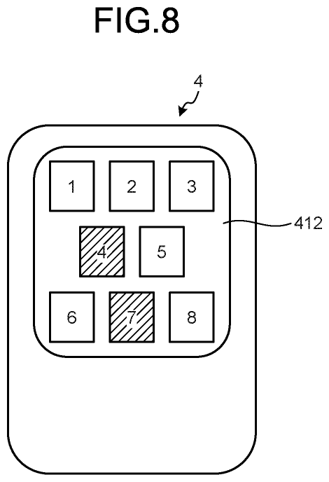

Next, a modification of the first embodiment of the disclosure is described. FIGS. 7 and 8 are diagrams illustrating an example of the notification mode by the receiving device 4 included in the capsule endoscope system 1 according to the modification of the first embodiment of the disclosure. The receiving device 4 illustrated in FIGS. 7 and 8 includes a touch panel provided on the display section 412.

According to the modification, at Step S106 of the above-described flowchart illustrated in FIG. 5, the output unit 408 first causes the display section 412 to display the message notifying that the receiving antennas have a poor attachment state (see FIG. Then, when the subject H or the user (medical personnel) presses the “Yes” button displayed on the display section 412, a transition is made to the screen indicating the attachment state of each of the receiving antennas as illustrated in FIG. 8. When the subject H or the user presses the “No” button displayed on the display section 412, for example, the notification image is hidden.

When a transition is made to the screen illustrated in FIG. 8, the light for the number of the receiving antenna having a poor attachment state is turned on (in FIG. 8, the number 4 and the number 7). The medical personnel checks the screen illustrated in FIG. 8 to check the receiving antenna having a poor attachment state and then handle the situation, for example, reattaches the receiving antenna. In the case described according to the present modification (FIG. 8), the indication is displayed with the number assigned to the receiving antenna; however, a configuration may be such that, in a case where a different unique color is assigned to each receiving antenna, the light in the color is emitted in accordance with the attachment state.

### Second Embodiment

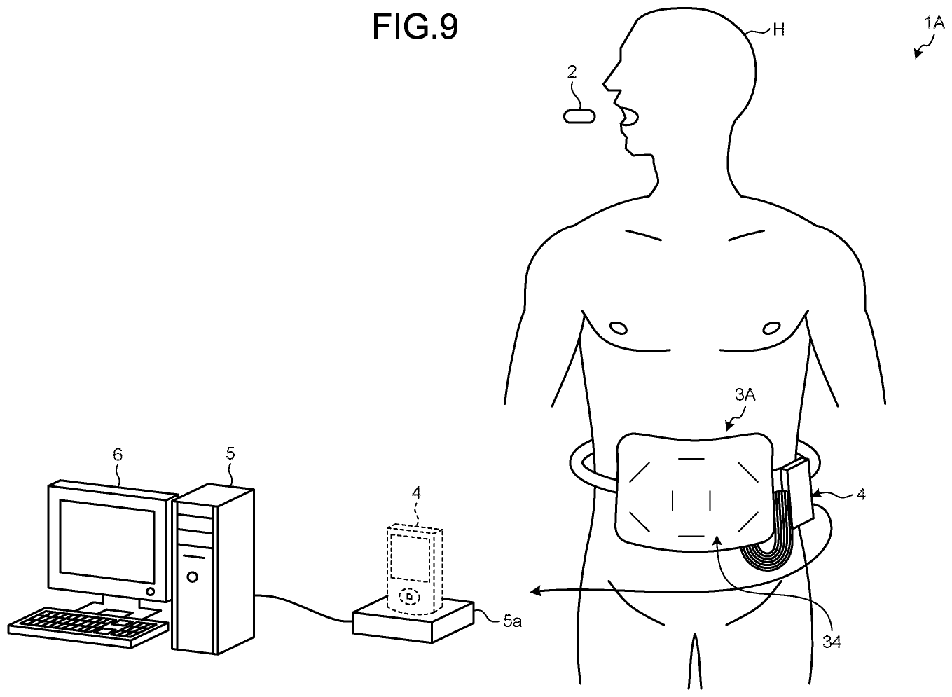

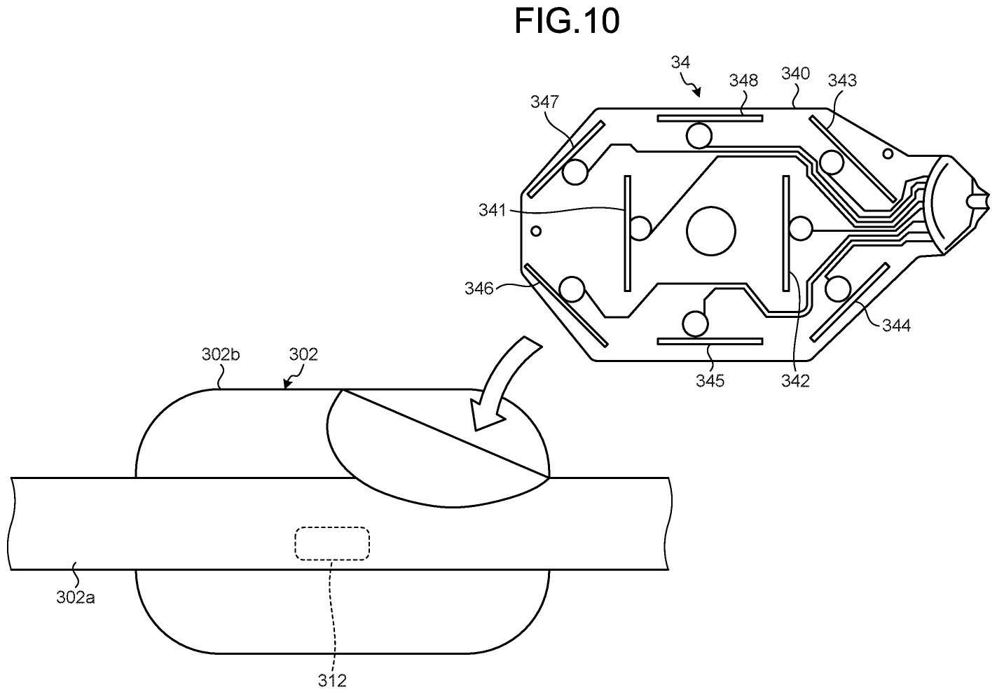

Next, a second embodiment of the disclosure is described. In the second embodiment, only the difference from the first embodiment described above is described. FIG. 9 is a schematic view illustrating a schematic configuration of a capsule endoscope system 1A according to the second embodiment. FIG. 10 is a diagram illustrating a schematic configuration of a receiving antenna included in the capsule endoscope system 1A according to the second embodiment. The capsule endoscope system 1A according to the second embodiment includes the capsule endoscope 2; a receiving antenna unit 3A including a board member 34 having a plurality of receiving antennas 341 to 348 formed thereon and attached to the subject H and a detecting unit 312 that detects the pressure applied to the body surface by the receiving antennas 341 to 348 when the board member 34 is attached to the body surface; the receiving device 4 that receives a wireless signal transmitted from the capsule endoscope 2 via the receiving antennas 341 to 348 attached to the subject H; and the processing device 5. In the second embodiment, the receiving antenna unit 3A having a configuration different from that in the first embodiment is described.

The board member 34 includes a flexible board 340 having the receiving antennas 341 to 348 provided to form the antenna pattern that receives a wireless signal from the capsule endoscope 2. The receiving antennas 341 to 348 are coupled to the receiving device 4 via a cable (not illustrated) extending from the board member 34.

The detecting unit 312 converts the detected pressure into an electrical signal to generate pressure information and outputs the pressure information to the receiving device 4. The detecting unit 312 is configured by using a piezoelectric element or a load cell.

In the receiving antenna unit 3A, the above-described board member 34 is held by an antenna holder (securing part) 302. The antenna holder 302 includes: a belt section 302a that secures the antenna holder 302 to the subject H; and an antenna attaching portion 302b that is supported by the belt section 302a and has the board member 34 and the detecting unit 312 attached thereto.

The antenna attaching portion 302b is made of a cloth, or the like, and is shaped like a bag. The antenna attaching portion 302b houses the board member 34 and the detecting unit 312. The antenna attaching portion 302b has an opening that is formed by using, for example, a snap fastener, a zip fastener, or a hook and loop fastener so as to be opened and closed.

The detecting unit 312 is attached to the antenna attaching portion 302b at the position to be supported by the belt section 302a. Specifically, the detecting unit 312 is provided between the belt section 302a and the body surface of the subject H. The attachment position of the detecting unit 312 may be any position that does not interfere with the radio communication by the receiving antennas 341 to 348. Instead of being attached, the detecting unit 312 may be housed in a pocket provided in the antenna attaching portion 302b or may be provided integrally with the antenna attaching portion 302b.

According to the second embodiment, as is the case with the first embodiment, it is possible to detect the pressure to the subject H, determine the attachment state of the receiving antenna to the subject H, and notify the attachment state. According to the second embodiment, the detecting unit 312 determines whether the board member 34 is properly attached to the subject H. That is, the pressure detected by the detecting unit 312 is used to determine whether the receiving antennas 341 to 348, which is regarded as a single antenna, which includes, properly attached to the subject H.

According to the second embodiment, at Step S101 in the flowchart illustrated in FIG. 5, the single pressure is acquired as the pressure information on the receiving antennas 341 to 348 and, at Step S102, the analysis is performed for the board member 34 (the receiving antennas 341 to 348). Therefore, at Step S103, it is determined that all the receiving antennas are properly attached when the acquired single pressure is more than the threshold (Step S102: Yes), and it is determined that no receiving antennas are properly attached (Step S103: No) when the acquired single pressure is less than the threshold even if some of the receiving antennas is properly attached.

According to the above-described second embodiment, the detecting unit 312 is provided in the antenna holder 302 to which the board member 34 having the receiving antennas 341 to 348 formed thereon is attached so as to detect the pressure against the subject H, and the receiving device 4 conducts analysis as to whether the receiving antenna (the board member 34) is properly attached in accordance with the result of detection by the detecting unit 312. Thus, it is possible to detect whether a receiving antenna is properly secured to the subject H. According to the second embodiment, the subject H or the user is prompted to properly attach the receiving antenna before examination, and therefore the examination by the capsule endoscope 2 may be executed while the receiving antenna is properly attached.

### Modification of the Second Embodiment

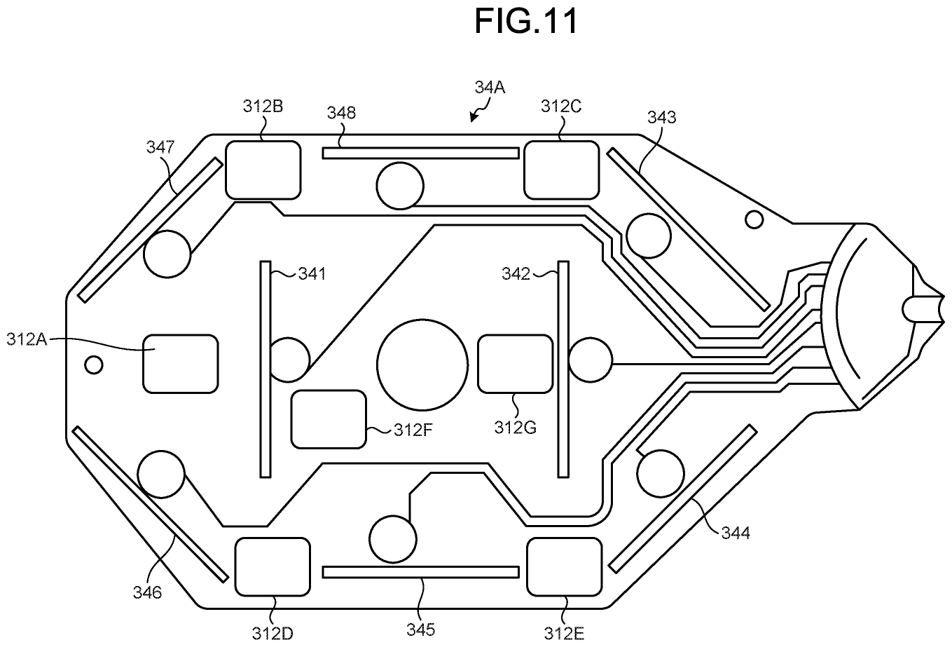

Next, a modification of the second embodiment of the disclosure is described. FIG. 11 is a diagram illustrating a schematic configuration of the receiving antennas included in a capsule endoscope system according to a modification of the second embodiment of the disclosure. According to the modification, instead of the board member 34, a board member 34A is included in the configuration according to the second embodiment described above. The board member 34A having a configuration different from that in the second embodiment is described below.

The board member 34A includes: the receiving antennas 341 to 348 that form the antenna pattern to receive a wireless signal from the capsule endoscope 2; and a plurality of detecting units (detecting units 312A to 312G).

Each of the detecting units 312A to 312G converts the detected pressure into an electrical signal to generate the pressure information and outputs the pressure information to the receiving device 4. Each of the detecting units 312A to 312G is located near any one of the receiving antennas 341 to 348 at a position where the detecting units 312A to 312G are not overlapped with each other. The detecting units 312A to 312G are configured by using a piezoelectric element or a load cell.

According to the present modification, as is the case with the first embodiment, it is possible to detect the pressure against the subject H, determine the attachment state of the receiving antenna to the subject H, and notify the attachment state. According to the present modification, it is determined whether the receiving antennas 341 to 348 are properly attached to the subject H in accordance with the detection results of the detecting units 312A to 312G. Specifically, it is determined whether the receiving antenna is properly attached to the subject H based on the combination of the detecting units that detect the pressure less than the threshold among the detecting units 312A to 312G. With regard to the combination of detecting units, for example, the storage unit 409 previously stores the table in which the pair of detecting units is related to the receiving antenna having a poor attachment state. For example, when the pressures detected by the detecting unit 312B and the detecting unit 312C are less than the threshold, the identifying unit 405 identifies the receiving antenna 348 sandwiched between the detecting unit 312B and the detecting unit 312C as a receiving antenna having a poor attachment state.

According to the above-described modification, detecting units 312A to 312G are provided, and the receiving device 4 conducts analysis as to whether the receiving antenna is properly attached or which receiving antenna is not properly attached in accordance with the result of detection by the detecting units 312A to 312G. Thus, it is possible to detect whether the receiving antenna is properly secured to the subject. According to the present modification, the subject H or the user is prompted to properly attach the receiving antenna before examination, and therefore the examination by the capsule endoscope 2 may be executed while the receiving antenna is properly attached.

### Third Embodiment

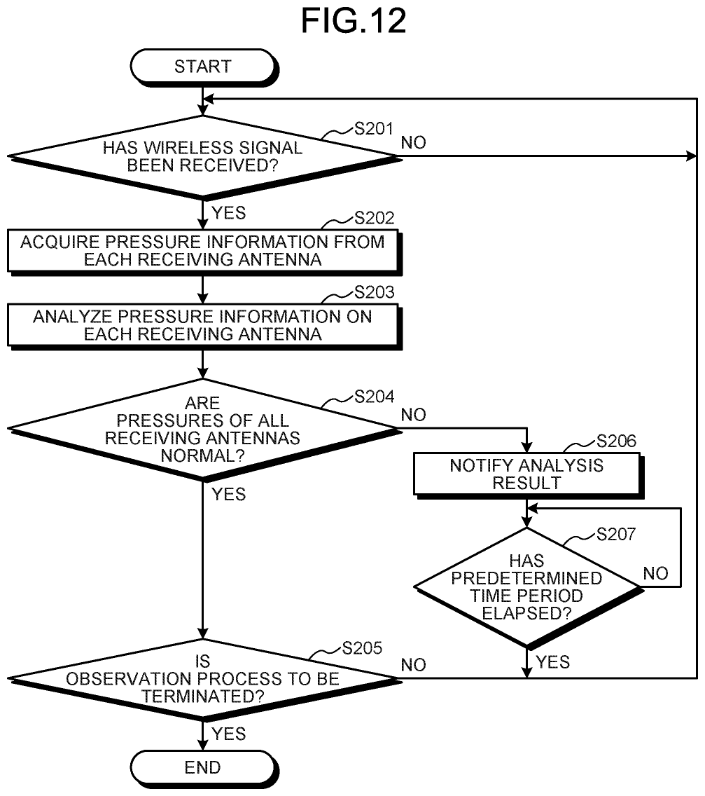

Next, a third embodiment of the disclosure is described. In the third embodiment, only the difference from the first embodiment described above is described. As the configuration of the capsule endoscope system is the same as that in the above-described first embodiment, the description thereof is omitted. FIG. 12 is a flowchart illustrating an antenna attachment checking process performed by the capsule endoscope system according to the third embodiment of the disclosure. In the third embodiment, the attachment state of the receiving antenna is checked during the examination (during the process to acquire image data) performed by the capsule endoscope 2, that is, when the receiving antenna receives a wireless radio.

When a wireless signal has been received from the capsule endoscope 2 (Step S201: Yes), the control unit 410 proceeds to Step S202. When no wireless signal has been received from the capsule endoscope 2 (Step S201: No), the control unit 410 repeatedly checks whether a wireless signal has been received.

At Step S202, the detecting unit 321 generates pressure information, and the pressure information acquiring unit 403 acquires the pressure information generated by the detecting unit 321. The pressure information acquiring unit 403 acquires the pressure information that is generated for each of the receiving antennas 3a to 3h and that is detected when a wireless signal is received.

Then, the determining unit 404 and the identifying unit 405 analyze the pressure information (Step S203). In the same manner as that at Step S102 described above, the determining unit 404 determines whether there is a receiving antenna whose pressure is less than the threshold based on the pressure information on each of the receiving antennas. Then, when the determining unit 404 determines that there is a receiving antenna whose pressure is less than the threshold, the identifying unit 405 identifies the corresponding receiving antenna based on the pressure information. The identifying unit 405 outputs, to the control unit 410, the analysis result in which the identified receiving antenna is related to the information indicating that there is a receiving antenna whose pressure is not appropriate. Conversely, when it is determined that there is no receiving antenna whose pressure is less than the threshold, the identifying unit 405 outputs, to the control unit 410, the analysis result that is the information indicating that the pressures of all the receiving antennas are normal.

At Step S204 after Step S203, the control unit 410 determines whether the pressures of all the receiving antennas are normal based on the analysis result. When it is determined that the pressures of all the receiving antennas are normal in accordance with the analysis result (Step S204: Yes), the control unit 410 proceeds to Step S205.

At Step S205, the control unit 410 determines whether the observation process by the capsule endoscope 2 is to be terminated. When a wireless signal is input from the capsule endoscope 2 (Step S205: No), the control unit 410 returns to Step S201 to repeat the above-described process. Conversely, the control unit 410 terminates the attachment checking process when the instruction signal for terminating the observation is input or no wireless signal is input from the capsule endoscope 2 after the elapse of the set time period (Step S205: Yes).

Conversely, at Step S204, when it is determined that there is a receiving antenna whose pressure is not normal in accordance with the analysis result (Step S204: No), the control unit 410 proceeds to Step S206.

At Step S206, the control unit 410 causes the output unit 408 to notify the analysis result. The output unit 408 generates sound and presents, on the display, the information indicating that the receiving antenna is not properly attached or causes the LED to emit light in the color indicating a poor attachment state.

At Step S207 after Step S206, the control unit 410 determines whether the predetermined time period has elapsed after the previous pressure information is acquired. When it is determined that the predetermined time period has not elapsed (Step S207: No), the control unit 410 repeatedly checks the elapsed time period. Conversely, when it is determined that the predetermined time period has elapsed (Step S207: Yes), the control unit 410 returns to Step S201 and repeats the above-described process.

As described above, even during the examination by the capsule endoscope 2, the attachment checking process is automatically performed, and the notification process is continued.

According to the above-described third embodiment, during the examination by the capsule endoscope 2, as is the case with the first embodiment, the detecting unit 321 is provided to detect the pressure against the subject with regard to each of the receiving antennas, and the receiving device 4 conducts analysis as to whether the receiving antenna is properly attached or which receiving antenna is not properly attached in accordance with the result of detection by the detecting unit 321. Thus, it is possible to detect whether a receiving antenna is properly secured to the subject. According to the third embodiment, the subject H or the user is prompted to properly attach the receiving antenna during the examination, and therefore the receiving antenna may be reattached properly and immediately in the case of a poor attachment state.

According to the above-described third embodiment, when it is determined that the pressures of all the receiving antennas are normal (Step S204: Yes), the control unit 410 may cause the output unit 408 to notify the analysis result.

According to the third embodiment, the control unit 410 may perform the above-described antenna attachment checking process each time a wireless signal is received, may perform the antenna attachment checking process by decimating sequentially transmitted wireless signals at preset receiving interval, or may perform the antenna attachment checking process when an instruction signal is input via the operating unit 406. In a case where the antenna attachment checking process is performed in accordance with an instruction via the operating unit 406, it is determined whether an instruction signal has been received instead of the above-described Step S201 so that the series or processes is performed.

The above-described third embodiment may be applied to the second embodiment. Specifically, in the configuration including the receiving antenna unit 3A according to the second embodiment, the attachment state of the receiving antenna may be checked during the examination (during the process to acquire image data) by the capsule endoscope 2.

### Fourth Embodiment

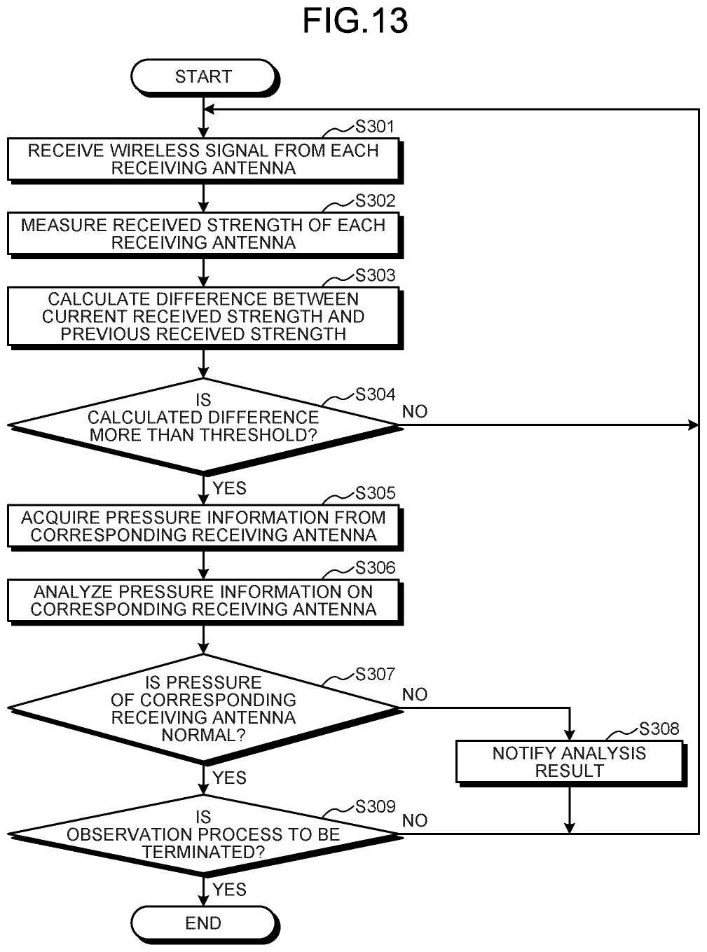

Next, a fourth embodiment of the disclosure is described. In the fourth embodiment, only the difference from the first embodiment described above is described. As the configuration of the capsule endoscope system is the same as that in the above-described first embodiment, the description thereof is omitted. FIG. 13 is a flowchart illustrating an antenna attachment checking process performed by the capsule endoscope system according to the fourth embodiment of the disclosure. According to the fourth embodiment, the attachment state of the receiving antenna is checked when a predetermined condition is satisfied during the examination (during the process to acquire image data) by the capsule endoscope 2.

When a wireless signal is received from the capsule endoscope 2 (Step S301), the control unit 410 proceeds to Step S302.

At Step S302, the received strength measuring unit 402 measures the received strength of a wireless signal with respect to each of the receiving antennas 3a to 3h when the receiving unit 401 receives the wireless signal. With regard to the received strength measured by the received strength measuring unit 402, the storage unit 409 stores at least the two latest measurement results in chronological order.

At Step S303 after Step S302, the control unit 410 calculates the difference between the currently measured received strength and the previously measured received strength. In a case where the antenna attachment checking process is performed by decimating wireless signals at a receiving interval, the difference between two received strengths adjacent to each other in chronological order among the received strengths extracted after the decimation is calculated.

Then, the control unit 410 determines whether the calculated difference is more than a preset threshold (Step S304). The threshold is a value that is set to determine whether the attachment state of a receiving antenna is to be checked. When it is determined that the calculated difference is less than the threshold (Step S304: No), the control unit 410 proceeds to Step S301. Conversely, when it is determined that the calculated difference is more than the threshold (Step S304: Yes), the control unit 410 proceeds to Step S305.

At Step S305, the detecting unit 321 generates pressure information, and the pressure information acquiring unit 403 acquires the pressure information generated by the detecting unit 321. Here, the pressure information acquiring unit 403 acquires the pressure information that belongs to the receiving antenna of which the difference between the received strengths is more than the threshold among the receiving antennas 3a to 3h and that is detected when the wireless signal whose received strength is measured is received.

Then, the determining unit 404 and the identifying unit 405 analyze the pressure information with regard to the receiving antenna whose pressure information has been acquired (Step S306). The determining unit 404 determines whether the pressure of the receiving antenna is less than the threshold. When it is determined that the pressure is less than the threshold, the identifying unit 405 identifies the corresponding receiving antenna as a receiving antenna having a poor attachment state. The identifying unit 405 outputs, to the control unit 410, the analysis result indicating that the corresponding receiving antenna has a poor attachment state. Conversely, when it is determined that there is no receiving antenna having a pressure less than the threshold with regard to the acquired pressure information, the identifying unit 405 outputs, to the control unit 410, the analysis result that. is the information indicating that the pressures of all the receiving antennas are normal.

At Step S307 after Step S306, the control unit 410 determines whether the pressure of the corresponding receiving antenna is normal based on the analysis result. When it is determined that the pressure of the corresponding receiving antenna is not normal as a result of the analysis (Step S307: No), the control unit 410 proceeds to Step S308.

At Step S308, the control unit 410 causes the output unit 408 to notify the analysis result. The output unit 408 generates sound and presents, on the display, the information indicating that the receiving antenna is not properly attached or causes the LED to emit light in the color indicating a poor attachment state.

Conversely, at Step S307, when it is determined that the pressure of the corresponding receiving antenna is normal as a result of the analysis (Step S307: Yes), the control unit 410 proceeds to Step S309.

At Step S309, the control unit 410 determines whether the observation process by the capsule endoscope 2 is to be terminated. When a wireless signal is input from the capsule endoscope 2 (Step S309: No), the control unit 410 returns to Step S301 to repeat the above-described process. Conversely, the control unit 410 terminates the attachment. checking process when the instruction signal for terminating the observation is input or no wireless signal is input from the capsule endoscope 2 after the elapse of the set time period (Step S309: Yes).

As described above, even during the examination by the capsule endoscope 2, the attachment checking process is automatically performed with regard to the corresponding receiving antenna in accordance with a change in the received strength, and the notification process is performed in accordance with an analysis result.

According to the above-described fourth embodiment, during the examination by the capsule endoscope 2, as is the case with the first embodiment, the detecting unit 321 is provided to detect the pressure against the subject with regard to each of the receiving antennas, and the receiving device 4 conducts analysis as to whether the receiving antenna is properly attached or which receiving antenna is not properly attached in accordance with the result of detection by the detecting unit 321. Thus, it is possible to detect whether a receiving antenna is properly secured to the subject. According to the fourth embodiment, the subject H or the user is prompted to properly attach the receiving antenna during the examination, and therefore the receiving antenna may be reattached properly and immediately in the case of a poor attachment state.

The above-described fourth embodiment may be applied to the second embodiment. Specifically, in the configuration including the receiving antenna unit 3A according to the second embodiment, the attachment state of the receiving antenna may be checked based on a difference between the received strengths during the examination (during the process to acquire image data) by the capsule endoscope 2.

### Fifth Embodiment

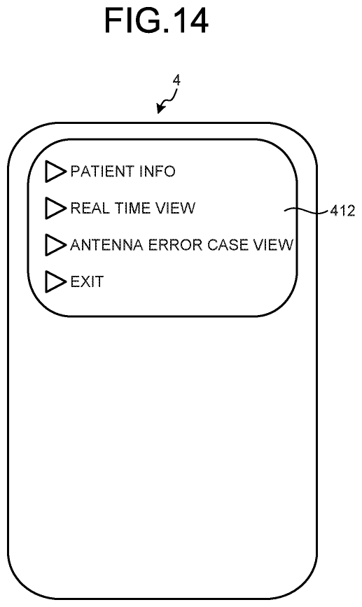

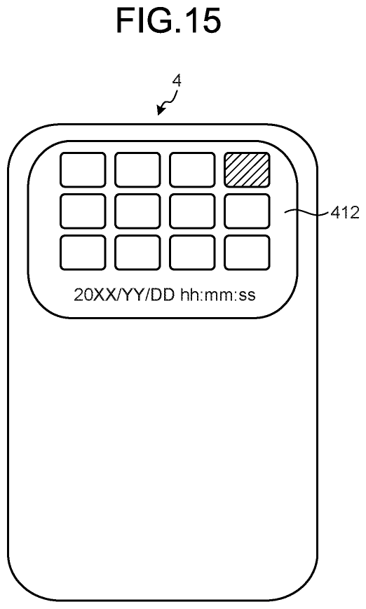

Next, a fifth embodiment of the disclosure is described. In the fifth embodiment, only the difference from the first embodiment described above is described. As the configuration of the capsule endoscope system is the same as that in the above-described first embodiment, the description thereof is omitted, FIGS. 14 and 15 are diagrams illustrating an example of the notification mode by the receiving device 4 included in the capsule endoscope system according to the fifth embodiment of the disclosure. According to the fifth embodiment, the attachment state of the receiving antenna is checked on the display of the receiving device 4 during the examination or after the examination by the capsule endoscope 2.

For example, during the examination (imaging process) by the capsule endoscope 2, the display section 412 of the receiving device 4 displays a selection menu for selecting the information to be displayed (see FIG. 14). FIG. 14 illustrates the selection menu representing patient information, real-time image display, receiving antenna error display, or the termination of the selection menu screen. A touch panel is provided on the display section 412 so that the user may press the information to be displayed to select it.

In a case where the user selects the receiving antenna error display, the list of images acquired by the receiving antenna whose attachment state is determined to be poor is displayed (see FIG. 15). When an image is selected on the screen, the date and time of the acquired image is displayed below the list of images.

According to the above-described fifth embodiment, when it is determined that the receiving antenna has a poor attachment state, the image acquired by the receiving antenna is checked by the receiving device 4 so that the effect on the image due to the receiving antenna may be checked.

### Sixth Embodiment

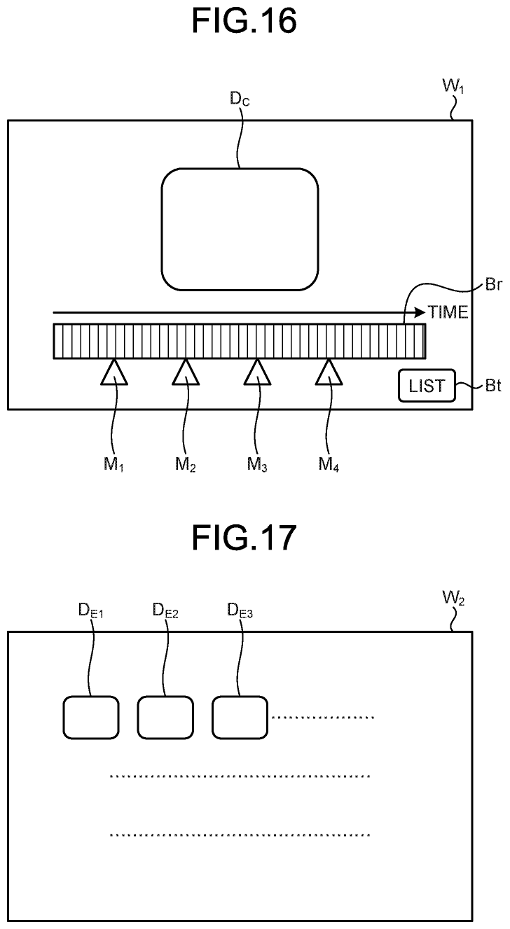

Next, a sixth embodiment of the disclosure is described. In the sixth embodiment, only the difference from the first embodiment described above is described. As the configuration of the capsule endoscope system is the same as that in the above-described first embodiment, the description thereof is omitted. FIGS. 16 and 17 are diagrams illustrating an example of the display screen of the display device included in the capsule endoscope system according to the sixth embodiment of the disclosure. According to the sixth embodiment, after the end of the examination by the capsule endoscope 2, the attachment state of the receiving antenna is checked with the display device 6. According to the sixth embodiment, the storage unit 56 stores the pressure detected by the detecting unit 321, the wireless signal acquired from the receiving device 4, and the analysis result by the determining unit 404 or the identifying unit 405 in association with one another.

For example, after the end of the examination by the capsule endoscope 2, the display device 6 presents a display image W₁ representing any selected captured image D_{c}, a display bar Br, and a list display button Bt under the control of the display controller 54. The display bar Br presents, for example, straight lines corresponding to the respective images acquired from the capsule endoscope 2 at equal intervals in chronological order of imaging. The straight line is displayed in the color corresponding to the feature of an image. On the display bar Br, markers M, to M₄ are attached to the positions of the images corresponding to the receiving antennas that are identified. as receiving antennas having a poor attachment state based on analysis results. When the user selects the straight line corresponding to the marker M₁, for example, the image corresponding to the receiving antenna that is identified as a receiving antenna having a poor attachment state may be displayed as the captured image D_{c}.

When the user selects the list display button Bt, the display device 6 displays a display image W₂ representing the list of images D_{E1}, D_{E2}, D_{E3}, . . . , acquired by the receiving antennas when it is determined that the receiving antennas have a poor attachment state (see FIG. 17). This allows the user to check the image acquired by the receiving antenna that is identified as a receiving antenna having a poor attachment state.

According to the above-described sixth embodiment, when it is determined that the receiving antenna has a poor attachment state, the image acquired by the receiving antenna is checked by the display device 6 so that the effect on the image due to the receiving antenna may be checked.

Although the embodiments for carrying out the disclosure have been described above, the disclosure is not limited to only the embodiments and the modifications described above. The disclosure may include not only the embodiments and the modifications described above but also various embodiments without departing from the technical idea described in the scope of claims. The configurations in the embodiments and the modifications may be combined as appropriate.

A configuration may be such that the execution program corresponding to each process performed by each component in the capsule endoscope 2, the receiving antenna units 3, 3A, the receiving device 4, and the processing device 5 in the capsule endoscope systems 1, 1A according to the first embodiment to the sixth embodiment is provided by being recorded, in the form of a file that is installable or executable, in a recording medium readable by a computer, such as a CD-ROM, a flexible disk (FD), a CD-R, or a DVD, or may be stored in a computer connected via a network such as the Internet to be provided by being downloaded via the network. Furthermore, a configuration may be such that the execution program may be provided or distributed via a network such as the Internet.

### INDUSTRIAL APPLICABILITY

As described above, the receiving unit and processing system according to the disclosure are useful in detecting whether the receiving antenna is properly secured to the subject.

According to the disclosure, there is an advantage such that it is possible to detect whether the receiving antenna is properly secured to the subject.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A receiving unit comprising:
a receiving antenna configured to receive a wireless signal transmitted from a capsule endoscope introduced into a subject, the receiving antenna being attached to a body surface of the subject;
a detector configured to detect a pressure between the receiving antenna and the subject; and
a processor comprising hardware, the processor being configured to
determine an attachment state of the receiving antenna to the body surface based on the pressure, and
output a determination result of the attachment state.

2. The receiving unit according to claim 1, wherein the detector is provided on a surface of the receiving antenna facing the body surface.

3. The receiving unit according to claim 1, further comprising a securing part configured to secure the receiving antenna to the body surface, wherein
the detector is provided inside the securing part and at a side closer to the body surface than the receiving antenna.

4. The receiving unit according to claim 1, wherein the processor is configured to determine the attachment state when power of the receiving unit is turned on.

5. The receiving unit according to claim 1, wherein the processor is configured to determine the attachment state when the receiving antenna receives the wireless signal.

6. The receiving unit according to claim 1, wherein the processor is configured to determine the attachment state when an input instruction signal for starting determination on the attachment state is received.

7. The receiving unit according to claim 1, wherein the processor is further configured to measure a received strength of the wireless signal, and determine the attachment state when a difference between two received strengths that are adjacent to each other in chronological order is more than a preset threshold.

8. The receiving unit according to claim 1, further comprising:
a storage configured to store the pressure and the wireless signal in association with each other, wherein
the processor is further configured to identify the wireless signal corresponding to an acquisition time of a pressure for which the processor determines a poor attachment state.

9. The receiving unit according to claim 8, further comprising:
a display configured to display a list of images obtained from the wireless signals identified by the processor.

10. The receiving unit according to claim 1, wherein
the receiving antenna includes a plurality of receiving antennas, and
the processor is configured to
determine an attachment state of each of the receiving antennas, and
output a determination result of the attachment state in an identifiable manner.

11. The receiving unit according to claim 3, wherein
the receiving antenna includes a plurality of receiving antennas,
the receiving unit further includes a board on which the receiving antennas are mounted, and
the securing part includes:
a holding portion configured to hold the board and the detector; and
an attaching portion configured to attach the holding portion to the body surface.

12. The receiving unit according to claim wherein
the receiving antenna includes a plurality of receiving antennas,
the detector includes a plurality of detectors,
the receiving unit further includes a board on which the receiving antennas and the detectors are mounted at different positions, and
the processor is configured to determine an attachment state of each of the receiving antennas to the body surface based on pressures detected by the detectors.

13. A processing system comprising:
a receiving unit configured to receive a wireless signal transmitted from a capsule endoscope introduced into a subject to acquire an image; and
a processing device configured to process the image,
the receiving unit including:
a receiving antenna configured to receive the wireless signal, the receiving antenna being attached to a body surface of the subject;
a detector configured to detect a pressure between the receiving antenna. and the subject; and
a processor comprising hardware, the processor being configured to
determine an attachment state of the receiving antenna to the body surface based on the pressure, and
identify the wireless signal corresponding to an acquisition time of a pressure for which the processor determines a poor attachment state, and
the processing device including:
a first storage configured to store the pressure, the image, and a determination result in association with one another, the pressure, the image, and the determination result being acquired from the receiving unit; and
a controller configured to cause a first display to display the determination result and the image corresponding to the determination result.

14. The processing system according to claim 13, wherein the processor is configured to determine the attachment state when power of the receiving unit is turned on.

15. The processing system according to claim 13, wherein the processor is configured to determine the attachment state when the receiving antenna receives the wireless signal.

16. The processing system according to claim 13, wherein the processor is configured to determine the attachment state when an input instruction signal for starting determination on the attachment state is received.

17. The processing system according to claim 13, wherein the processor is further configured to measure a received strength of the wireless signal, and determine the attachment state when a difference between two received strengths that are adjacent to each other in chronological order is more than a preset threshold.

18. The processing system according to claim 13, wherein the receiving unit further includes a second storage configured to store the pressure and the wireless signal in association with each other.

19. The processing system according to claim 18, further comprising:
a second display configured to display a list of images obtained from the wireless signals identified by the processor.

20. The processing system according to claim 13, wherein
the receiving antenna included in the receiving unit includes a plurality of receiving antennas,
the processor is configured to
determine an attachment state of each of the receiving antennas, and
output a determination result of the attachment state in an identifiable manner.
